(19) United States
(12) Patent Application Publication
(10) Pub. No.: US 2017/0146511 A1
(43) Pub. Date: May 25, 2017

(54) NANOGAP ELECTRODES WITH DISSIMILAR MATERIALS

(71) Applicant: Osaka University, Osaka (JP)

(72) Inventors: Masateru Taniguchi, Osaka (JP); Tomoji Kawai, Osaka (JP); Takahito Ohshiro, Osaka (JP)

(21) Appl. No.: 15/344,199

(22) Filed: Nov. 4, 2016

Related U.S. Application Data
(63) Continuation of application No. PCT/JP2015/063965, filed on May 8, 2015.
(60) Provisional application No. 61/990,527, filed on May 8, 2014.

(51) Int. Cl. G01N 33/487; C12Q 1/68; G01N 27/447

(52) U.S. Cl. CPC G01N 33/48721; G01N 27/44791; C12Q 1/6869

(57) ABSTRACT

The present disclosure provides devices, systems and methods for effectuating nanoelectrodes for use with determining the sequence of double stranded biopolymers. Various modified bases and different metals may be utilized alone or in combination so as to provide differentiation between different nucleobases and to determine which base is associated with which strand.

### CROSS-REFERENCE

This application claims priority to U.S. Provisional Patent Application Ser. No. 61/990,527, filed May 8, 2014, which is entirely incorporated herein by reference.

### DESCRIPTION OF THE RELATED ART

Nucleic acid sequencing is the process of determining the order of nucleotides within a nucleic acid molecule, such as deoxyribonucleic acid (DNA) or ribonucleic acid (RNA). The determination of the sequence of a nucleic acid molecule may provide various benefits, such as aiding in diagnosing and/or treating a subject. For example, the nucleic acid sequence of a subject may be used to identify, diagnose and potentially develop treatments for genetic diseases.

### SUMMARY OF THE INVENTION

While there are nucleic acid sequencing methods and systems presently available, recognized herein are various limitations associated with such systems. Double-stranded deoxyribonucleic acid (DNA) has been difficult to measure by using sequencers. Some DNA sequencing systems, including electrophoretic based Sanger systems, sequencing by synthesis approaches, and nanopore approaches, utilize single stranded target nucleic acids. In order to obtain single-stranded DNA, double-stranded DNA molecules have typically been heated and or placed in low ionic conditions or in highly denaturing solvents, such as formamide, in order to denature the nucleic acids. To facilitate denaturation sequencers routinely utilize an elevated temperature control device, complicating the system.

Nanopores may be useful for determining the sequence of a single stranded DNA strand, and may be utilized to detect double stranded nucleic acids, but have been unable to provide sequence information for double stranded nucleic acids. Tunneling nanoelectrodes associated with nanochannels may be utilized to provide sequence data for single stranded nucleic acids, but have been unable to provide useful information about double stranded nucleic acids as the tunneling systems have been unable to distinguish a base such as a guanine (G) in a first strand hybridized to a cytosine (C) in a second complementary strand from a C in a first strand hybridized to a G in a second complementary strand, and similarly have been unable to distinguish a base such as a thymine (T) in a first strand hybridized to an adenine (A) in a second complementary strand from an A in a first strand hybridized to a T in a second complementary strand. Additionally, some sequencing systems which utilize single stranded nucleic acids may need to provide an approach for addressing any secondary structure which may result from hybridization of parts of the nucleic acid strand to itself, providing further constraints on the system.

The present disclosure provides methods and apparatuses for creating nanoelectrode systems which may be used for sensing and/or sequencing a nucleic acid molecule, such as deoxyribonucleic acid (DNA) or ribonucleic acid (RNA), or sensing and/or sequencing other biopolymers and detecting and identifying other molecules.

An aspect of the present disclosure provides a system for detecting a sample polymer, comprising: an electrode structure, wherein the electrode structure includes at least one pair of nanoelectrodes and a nanogap between the nanoelectrodes, wherein the at least one pair of nanoelectrodes comprises a first electrode and a second electrode, the first electrode comprising a first conductive material and the second electrode comprising a second conductive material different from the first conductive material; a voltage source that applies a voltage to the nanogap between the at least one pair of nanoelectrodes; a translocation unit that moves the sample polymer through the nanogap between the pair of nanoelectrodes; a measurement unit coupled to the at least one pair of nanoelectrodes, wherein the measurement unit measures electrical current passing through the sample polymer between the at least one pair of nanoelectrodes; and a computer processor coupled to the measurement unit and programmed to determine an orientation and identity of monomers of the sample polymer relative to the nanoelectrodes in accordance with the electrical current measured with the measurement unit.

In some embodiments of aspects provided herein, the first conductive material has a Fermi level different from a Fermi level of the second conductive material. In some embodiments of aspects provided herein, the first conductive material comprises gold and the second conductive material comprises silver. In some embodiments of aspects provided herein, the first conductive material comprises platinum and the second conductive material comprises silver. In some embodiments of aspects provided herein, the sample polymer is a biopolymer. In some embodiments of aspects provided herein, the sample polymer comprises a double-stranded nucleic acid. In some embodiments of aspects provided herein, the double-stranded nucleic acid is double-stranded deoxyribonucleic acid. In some embodiments of aspects provided herein, the sample polymer has one or more modified base types incorporated in one of the strands of the sample polymer. In some embodiments of aspects provided herein, the sample polymer includes one or more modified base types incorporated in one of the strands, wherein a molecule-electrode coupling for a modified base is different than for an unmodified base. In some embodiments of aspects provided herein, a width of the nanogap between the pair of nanoelectrodes is less than a diameter of a sample polymer. In some embodiments of aspects provided herein, the translocation unit is a pressure or electrokinetic source. In some embodiments of aspects provided herein, the pressure source is a positive pressure source. In some embodiments of aspects provided herein, the pressure source is a negative pressure source. In some embodiments of aspects provided herein, the electrical current comprises tunneling current.

Another aspect of the present disclosure provides a method for detecting a sample polymer, comprising: (a) subjecting the sample polymer to flow through a channel having an electrode structure, wherein the electrode structure includes at least one pair of nanoelectrodes and a nanogap between the nanoelectrodes, wherein the at least one pair of nanoelectrodes comprises a first electrode and a second electrode, the first electrode comprising a first conductive material and the second electrode comprising a second conductive material different from the first conductive material; (b) applying a voltage to the nanogap between the at least one pair of nanoelectrodes; (c) using a measurement unit coupled to the at least one pair of nanoelectrodes to measure electrical current passing through the sample polymer upon flow of the sample polymer through the channel and the nanogap; and (d) using a computer processor to determine an orientation and identity of monomers of the sample polymer relative to the nanoelectrodes in accordance with the electrical current measured with the measurement unit.

In some embodiments of aspects provided herein, the first conductive material has a Fermi level different from a Fermi level of the second conductive material. In some embodiments of aspects provided herein, the first conductive material comprises gold and the second conductive material comprises silver. In some embodiments of aspects provided herein, the first conductive material comprises platinum and the second conductive material comprises silver. In some embodiments of aspects provided herein, the sample polymer is a biopolymer. In some embodiments of aspects provided herein, the sample polymer comprises a double-stranded nucleic acid. In some embodiments of aspects provided herein, the double-stranded nucleic acid is double-stranded deoxyribonucleic acid. In some embodiments of aspects provided herein, the sample polymer has one or more modified base types incorporated in one of the strands of the sample polymer. In some embodiments of aspects provided herein, the sample polymer includes one or more modified base types incorporated in one of the strands, wherein a molecule-electrode coupling for a modified base is different than for an unmodified base. In some embodiments of aspects provided herein, a width of the nanogap between the pair of nanoelectrodes is less than a diameter of a sample polymer. In some embodiments of aspects provided herein, the translocation unit is a pressure or electrokinetic source. In some embodiments of aspects provided herein, the pressure source is a positive pressure source. In some embodiments of aspects provided herein, the pressure source is a negative pressure source. In some embodiments of aspects provided herein, the electrical current comprises tunneling current.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “Fig.” herein), of which:

FIG. 1 schematically illustrates a nanoelectrode gap with associated work functions associated with a hybridized nucleic acid base pair of a double stranded nucleic acid.

FIG. 2 schematically illustrates a nanoelectrode gap with associated work functions associated with a hybridized nucleic acid base pair of a double stranded nucleic acid with base paring orientation reversed from FIG. 1.

FIG. 3 illustrates tunneling currents and dwell times for a base pair in two different orientations.

FIG. 4 schematically illustrates potential steps in a nano gap tunneling current event utilizing nanoelectrodes with different metals.

FIG. 5 schematically illustrates potential steps in a nano gap tunneling current event utilizing nanoelectrodes with different metals with a reversed current path from FIG. 4.

FIG. 6 schematically illustrates electron transfer from a nanoelectrode to a base and the energy levels associated with a base pair.

FIG. 7 schematically illustrates potential steps in a nano gap tunneling current event utilizing nanoelectrodes with different metals.

FIG. 8 schematically illustrates potential steps in a nano gap tunneling current event utilizing nanoelectrodes with different metals with a reversed base orientation from FIG. 7.

FIGS. 9A-9C schematically illustrate a nanogap structure with dissimilar electrodes, the Fermi levels associated with the electrodes, the molecule-electrode coupling levels associated with the nucleobases, and the energy level shifts of an electron passing from one nanoelectrode to the other.

FIG. 10 illustrates a histogram of tunneling currents for single stranded DNA.

FIG. 11 illustrates a histogram of tunneling currents for double stranded DNA with different base pairing orientations.

FIG. 12 illustrates a histogram of tunneling currents for single stranded dCMP and methylated dCMP.

FIG. 13 illustrates a histogram of tunneling currents for single stranded DNA dGMP and oxo-dGMP.

FIG. 14 illustrates possible combinations of natural bases and modified bases.

FIG. 15 schematically illustrates a device with more than two nanoelectrodes associated with a single base interrogation region.

FIG. 16 schematically illustrates the energy levels of GC nucleobase pair.

FIGS. 17A-17B schematically illustrate energy states associated with electron tunneling of different orientations of a GC nucleobase pair.

FIG. 18 schematically illustrates a computer system that is programmed or otherwise configured to implement devices, systems and methods of the present disclosure.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “gap,” as used herein, generally refers to a pore, channel or passage formed or otherwise provided in a material. The material may be a solid state material, such as a substrate. The gap may be disposed adjacent or in proximity to a sensing circuit or an electrode coupled to a sensing circuit. In some examples, a gap has a characteristic width or diameter on the order of 0.1 nanometers (nm) to about 1000 nm. A gap having a width on the order of nanometers may be referred to as a “nano-gap” (also “nanogap” herein). In some situations, a nano-gap has a width that is from about 0.1 nanometers (nm) to 50 nm, 0.5 nm to 30 nm, or 0.5 nm or 10 nm, 0.5 nm to 5 nm, or 0.5 nm to 2 nm, or no greater than 2 nm, 1 nm, 0.9 nm, 0.8 nm, 0.7 nm, 0.6 nm, or 0.5 nm. In some cases, a nano-gap has a width that is at least about 0.5 nm, 0.6 nm, 0.7 nm, 0.8 nm, 0.9 nm, 1 nm, 2 nm, 3 nm, 4 nm, or 5 nm. In some cases, the width of a nano-gap can be less than a diameter of a biomolecule or a subunit (e.g., monomer) of the biomolecule.

The term “electrode,” as used herein, generally refers to a material or part that can be used to measure electrical current. An electrode (or electrode part) can be used to measure electrical current to or from another electrode. In some situations, electrodes can be disposed in a channel (e.g., nanogap) and be used to measure the current across the channel. The current can be a tunneling current. Such a current can be detected upon the flow of a biomolecule (e.g., protein) through the nano-gap. In some cases, a sensing circuit coupled to electrodes provides an applied voltage across the electrodes to generate a current. As an alternative or in addition to, the electrodes can be used to measure and/or identify the electric conductance associated with a biomolecule (e.g., an amino acid subunit or monomer of a protein). In such a case, the tunneling current can be related to the electric conductance.

The term “biomolecule,” as used herein generally refers to any biological material that can be interrogated with an electrical current and/or potential across a nano-gap electrode. A biomolecule can be a nucleic acid molecule, protein, or carbohydrate. A biomolecule can include one or more subunits, such as nucleotides or amino acids.

The term “nucleic acid,” as used herein, generally refers to a molecule comprising one or more nucleic acid subunits. A nucleic acid may include one or more subunits selected from adenosine (A), cytosine (C), guanine (G), thymine (T) and uracil (U), or variants thereof. A nucleotide can include A, C, G, T or U, or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is deoxyribonucleic acid (DNA) or ribonucleic acid (RNA), or derivatives thereof. A nucleic acid may be single-stranded or double stranded.

The term “protein,” as used herein, generally refers to a biological molecule, or macromolecule, having one or more amino acid monomers, subunits or residues. A protein containing 50 or fewer amino acids, for example, may be referred to as a “peptide.” The amino acid monomers can be selected from any naturally occurring and/or synthesized amino acid monomer, such as, for example, 20, 21, or 22 naturally occurring amino acids. In some cases, 20 amino acids are encoded in the genetic code of a subject. Some proteins may include amino acids selected from about 500 naturally and non-naturally occurring amino acids. In some situations, a protein can include one or more amino acids selected from isoleucine, leucine, lysine, methionine, phenylalanine, threonine, tryptophan and valine, arginine, histidine, alanine, asparagine, aspartic acid, cysteine, glutamine, glutamic acid, glycine, proline, serin and tyrosine.

The term “layer,” as used herein, refers to a layer of atoms or molecules on a substrate. In some cases, a layer includes an epitaxial layer or a plurality of epitaxial layers. A layer may include a film or thin film. In some situations, a layer is a structural component of a device (e.g., light emitting diode) serving a predetermined device function, such as, for example, an active layer that is configured to generate (or emit) light. A layer generally has a thickness from about one monoatomic monolayer (ML) to tens of monolayers, hundreds of monolayers, thousands of monolayers, millions of monolayers, billions of monolayers, trillions of monolayers, or more. In an example, a layer is a multilayer structure having a thickness greater than one monoatomic monolayer. In addition, a layer may include multiple material layers (or sub-layers). In an example, a multiple quantum well active layer includes multiple well and barrier layers. A layer may include a plurality of sub-layers. For example, an active layer may include a barrier sub-layer and a well sub-layer.

The term “adjacent” or “adjacent to,” as used herein, includes ‘next to’, ‘adjoining’, ‘in contact with’, and ‘in proximity to’. In some instances, adjacent to components are separated from one another by one or more intervening layers. For example, the one or more intervening layers can have a thickness less than about 10 micrometers (“microns”), 1 micron, 500 nanometers (“nm”), 100 nm, 50 nm, 10 nm, 1 nm, or less. In an example, a first layer is adjacent to a second layer when the first layer is in direct contact with the second layer. In another example, a first layer is adjacent to a second layer when the first layer is separated from the second layer by a third layer.

The term “substrate,” as used herein, refers to any workpiece on which film or thin film formation is desired. A substrate includes, without limitation, silicon, germanium, silica, sapphire, zinc oxide, carbon (e.g., graphene), SiC, AlN, GaN, spinel, coated silicon, silicon on oxide, silicon carbide on oxide, glass, gallium nitride, indium nitride, titanium dioxide and aluminum nitride, a ceramic material (e.g., alumina, AlN), a metallic material (e.g., molybdenum, tungsten, copper, aluminum), and combinations (or alloys) thereof. A substrate can include a single layer or multiple layers.

The term “LUMO,” as used herein, generally refers to the lowest unoccupied molecular orbital of a molecule.

The term “HOMO,” as used herein, generally refers to the highest occupied molecular orbital of a molecule.

A nanoelectrode configuration can comprise symmetrical nanoelectrodes with the same metals, in some cases gold, and is thus unable to differentiate between complementary base pairs for double stranded nucleic acids. A tunneling current detector cannot typically differentiate between a sequence in which one strand or a double stranded DNA is GGGG and another sequence in one strand of a double stranded DNA for which the sequence is GCGC, as both sequences have four pairs of GC or CG base pairs, which cannot be differentiated from each other.

Various methods for generating asymmetries by which the base orientation of the base pair under interrogation by tunneling current may be utilized to differentiate between the two possible base pair orientation.

In some embodiments, the bases may be modified in one strand and not the other strand of a double stranded nucleic acid, such that the molecule-electrode coupling levels of the modified bases may be different from the molecule-electrode coupling levels of the natural bases. In other embodiments the metals of the nanoelectrodes of a tunneling current device may be configured so as to comprise different metals, particularly at the tips of the nanoelectrodes, such that the Fermi and molecule-electrode coupling levels may not be symmetric with respect to the different orientations of the double stranded nucleic acid.

In some embodiments, a voltage source may be impressed across one or more nanoelectrode pairs, wherein the voltage across different nanoelectrode pairs may be a different voltage, and may particularly be different as a function of a nanogap spacing of metal pair which may be associated with a particular nanogap pair.

In some embodiments, several different nanoelectrode pairs may be utilized in a single channel, wherein some different electrode pairs may have different gap spacings, metal pair combinations, and wherein different electrode pairs may be utilized to detect different types of monomer pairings, including monomer pairings which comprise base modifications, by using different tunneling currents associated with different electrode pairs and different monomer pairs and orientations of said monomer pairs.

In some embodiments, a bias potential may be reversed while monitoring a monomer base pair, and different currents which may be associated with orientation and polarity of a bias field may be observed and utilized to at least in part determine the identity and orientation of a base pair. A bias field may be reversed at a rate which may be twice a nominal translocation speed of individual monomers relative to electrode pairs, or may be a reversed at a rate which is a higher integer multiple of a nominal translocation speed of individual monomers relative to electrode pairs, or bias field reversal may occur at a rate which may be more than twice a nominal translocation speed, but may be a noninteger multiple of a nominal translocation speed of individual monomers relative to electrode pairs.

A bias field may be reversed so as to have a symmetrical potential, or may be reversed in a manner such that a potential in one direction may be higher than a potential in another direction. A first period of time associated with a polarization of a bias field may be the same as a time associated with a period of second period of time wherein a bias field may be reversed relative to said first period of time, or may be shorter or longer. Periods of time associated with reversal may be uniform, or may be variable. Bias potential levels may be uniform, thus creating square waves, or may have rounded corners, or may have any other shape, such as a a sine wave, triangular saw tooth wave or other wave shapes.

In some embodiments, a measurement device (or measurement unit) may be provided to measure the tunneling current. The measurement device may comprise a transimpedance amplifier, an integrating amplifier, a current mirror, or any other appropriate current measurement or amplification approach, and an approach for quantifying the current, which may include an analog to digital converter (ADC), a delta sigma ADC, a flash ADC, a dual slope ADC, a successive approximation ADC, an integrating ADC, or any other appropriate type of ADC. The ADC may have a linear relationship between its output and the input, or may have an output which is tuned to the particular current levels which may be expected for a particular combination of bases, modified bases expected and metals utilized in a nanoelectrode pair. The response may be fixed, or may be adjustable, and may be adjustable particularly in conjunction with different outputs associated with the different nucleobases and or nucleobases modifications which may be utilized in an assay. The measuring device may measure the tunneling current which passes through a sample polymer, which may be a biopolymer as it passes through a nanogap of a nanoelectrode pair.

In some embodiments, a computer or other data processing device (e.g., computer processor) may be provided as a part of the system, wherein the computer may utilize measured data to determine the identity and or orientation of monomers of a polymer, which may be a biopolymer, relative to the nanoelectrodes which took the associated data. The computer or other data processing device may be computer which is incorporated into the device which includes the nanofluidics, or may be a computer which is incorporated within an instrument into which the nanofluidics device may be utilized, or may be an external device, which may be a cloud computing device.

A polymer can be translocated through a channel having at least one nanoelectrode pair using a translocation unit. Examples of translocation units include pumps and compressors. In some cases, the translocation unit can subject the polymer to flow using positive pressure. As an alternative, the translocation unit can subject the polymer to flow using negative pressure.

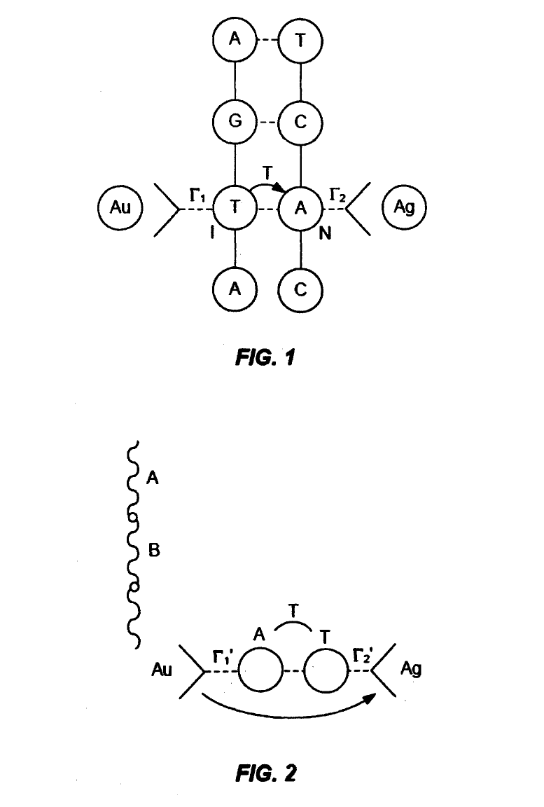

FIG. 1 schematically depicts a nanoelectrode configuration with two nanoelectrodes, wherein first nanoelectrode comprises a gold tip associated with a first Fermi level and a first molecule-electrode coupling level Γ₁, and a second nanoelectrode tip comprises a silver tip associated with a second Fermi level and a second molecule-electrode coupling level Γ₂. Current is depicted as passing from the first gold electrode to a T base associated with a Fermi level and a molecule-electrode coupling level Γ₁, then to a complementary A base associated with a molecular conduction T operator (where T(E)=V+VG(E)V), and then to a second silver tip associated with a second Fermi level and a second molecule-electrode coupling Γ₂.

FIG. 2 schematically depicts a nanoelectrode configuration with two nanoelectrodes wherein the orientation of the bases is reversed with respect to FIG. 1, wherein first nanoelectrode comprises a gold tip associated with a first Fermi level and a first molecule-electrode coupling level Γ₁′, and a second nanoelectrode tip comprises a silver tip associated with a second Fermi level and a second molecule-electrode coupling level Γ₂′. Current is depicted as passing from the first gold electrode to a T base associated with a first Fermi level and a first molecule-electrode coupling level Γ₁′, then to a complementary A base associated with a molecular conduction T operator (where T(E)=V+VG(E)V), and then to a second silver tip associated with a second Fermi level and a second molecule-electrode coupling level Γ₂′.

For a configuration as depicted in FIG. 1, a current I which may flow from the gold nanoelectrode to the T nucleobase, then to the A nucleobase, and then to the silver nanoelectrode (Au→T→A→Ag) is calculated to be: I∝Γ₁×T×Γ₂. For a configuration as depicted in FIG. 2, a current I′ which may flow from the gold nanoelectrode to the A nucleobase, then to the T nucleobase, and then to the silver nanoelectrode (Au→A→T→Ag) is calculated to be: I′∝Γ₂′×T×Γ₂′. As Γ1≠Γ₁′ and Γ2≠Γ₂′, I≠I′. These inequalities may thus permit a determination as to the orientation of the base pair.

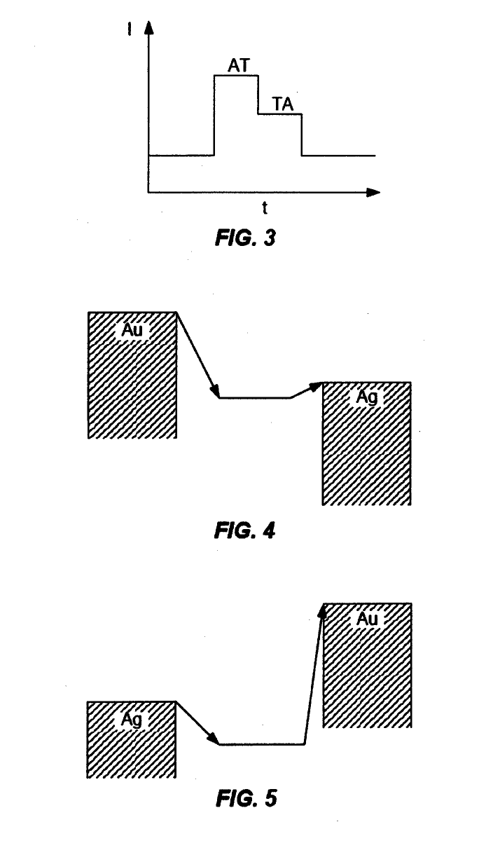

FIG. 3 illustrates potential difference in tunneling current and dwell time for two different base orientations with respect to a pair of nanoelectrodes with dissimilar metal tips, which thereby have different Fermi levels as described hereinabove. Similar plots may be generated for different base pairs wherein the one of the strands of a double stranded nucleic acid has modified bases, which may be naturally modified bases such as methylated cytosine bases, or may be synthetically modified bases. Systems utilizing modified bases may be differentiated using nanoelectrodes which wherein the tips comprise the same metal, or using nanoelectrodes wherein the tips comprise different metals. Both dwell times and currents may be different for different orientations and different base combinations, including base combinations with modified bases, such as methylated bases or oxo bases, and both dwell times and currents may be utilized to help determine the identity of bases, modifications of bases, and orientation of bases.

FIG. 4 schematically depicts an electron passing from a gold nanoelectrode to a lower energy state of a first nucleic acid base of a nucleic acid base pair wherein the passing of the electron from the gold nanoelectrode to the first nucleic acid base of a nucleic acid pair is associated with a first Fermi level and a first molecule-electrode coupling level Γ₁; the electron is then passed to a second nucleic acid base of a nucleic acid base pair using a molecular conduction T operator, and thence the electron passes to a second silver nanoelectrode which may be associated with a second Fermi level and a second molecule-electrode coupling level Γ₂.

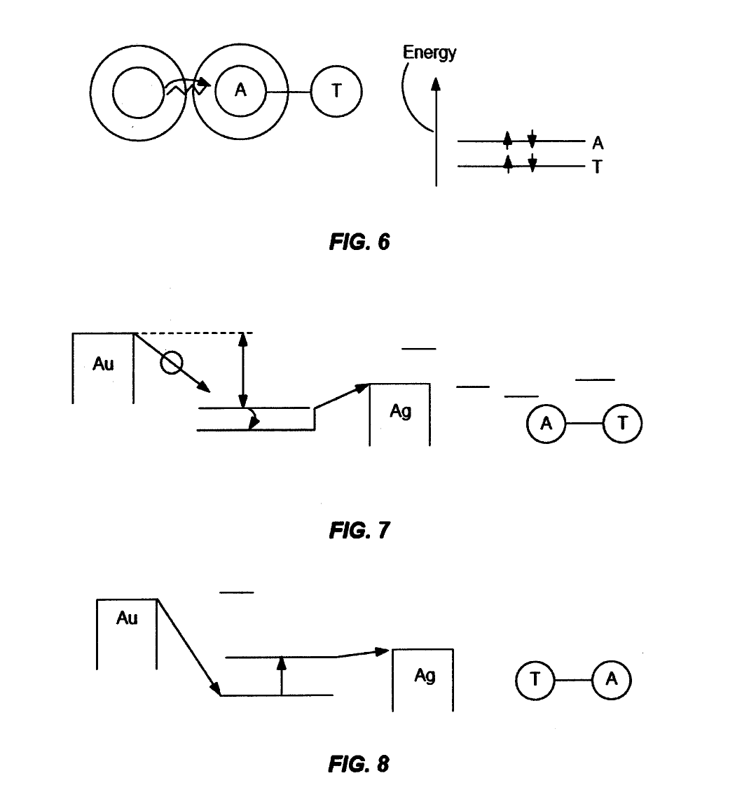

FIG. 5 schematically depicts an electron passing from a silver nanoelectrode to a lower energy state of a first nucleic acid base of a nucleic acid base pair wherein the passing of the electron from the silver nanoelectrode to the first nucleic acid base of a nucleic acid pair is associated with a first Fermi level and a molecule-electrode coupling level Γ₁′; the electron is then passed to a second nucleic acid base of a nucleic acid base pair using a molecular conduction T operator, and thence the electron passes to a second gold nanoelectrode which may be associated with a second Fermi level and a second molecule-electrode coupling level Γ₂′.

It can be seen that the change in energy states in FIG. 4 is quite different than the change in energy states in FIG. 5, and that the tunneling currents for systems with otherwise identical configurations with respect to gap spacings, nanoelectrode pair gap potentials, and nucleotide base pair. The resulting differences in tunneling current may be utilized to determine which base of a base pair is in which position relative to a nanoelectrode structure.

FIG. 6 schematically illustrates an energy state diagram depicting a nanoelectrode and a base pair, wherein the base pair is an AT nucleic acid base pair. The figure further depicts potential variations in energy levels of an electron associated with each nucleic acid base of the base pair, and potential shifts from one base of the nucleic acid base pair to the other base of the nucleic acid base pair.

FIG. 7 schematically depicts an energy state diagram associated with a tunneling current device wherein an electron passing from a gold nanoelectrode to a lower energy state of a first nucleic acid base (an A nucleobase) of a nucleic acid base pair. The energy state change associate with the transition of the electron from the gold nanoelectrode to the nucleic acid base pair associated with the first Fermi level and a first molecule-electrode coupling level Γ₁, which may be a HOMO associated with the A nucleobase, is depicted by a double headed arrow; the electron is then passed to a second nucleic acid base (a T nucleobase) of a nucleic acid base pair using a molecular conduction T operator, dropping to a lower energy state which may be a HOMO-1 associated with the T nucleobase, and thence the electron passes up to a higher energy state associated with a second silver nanoelectrode which may be associated with a second Fermi level and a second molecule-electrode coupling level Γ₂.

In a manner similar to that of FIG. 7, FIG. 8 schematically depicts an energy state diagram associated with a tunneling current device wherein an electron passing from a gold nanoelectrode to a lower energy state of a first nucleic acid base (an T nucleobase instead of an A nucleobase) of a nucleic acid base pair wherein the electron may have a molecule-electrode coupling to the HOMO-1 level of the first (T) nucleobase. The electron is then passed to a second nucleic acid base (an A nucleobase instead of a T nucleobase) of a nucleic acid base pair using a molecular conduction T operator, rising to a higher energy state which may be a HOMO level associated with the second (A) nucleobase, and thence the electron passes up to a higher energy state associated with a second silver nanoelectrode which may be associated with a second Fermi level Γ₂′ and a second molecule-electrode coupling.

As can be seen from inspecting the energy state changes associated with FIG. 7 and FIG. 8, there are distinct differences in the changes in energy level needed for an electron to transit from one nanoelectrode to another based on the orientation of the nucleobases of the nucleobase pair within the nanoelectrode pair with dissimilar metal tips. The resultant tunneling currents may thus be quite different, particularly as the second molecule-electrode coupling level Γ₂ of FIG. 7 is much higher and thus thermodynamically disadvantageous relative to the second molecule-electrode coupling level Γ₂′ of FIG. 8.

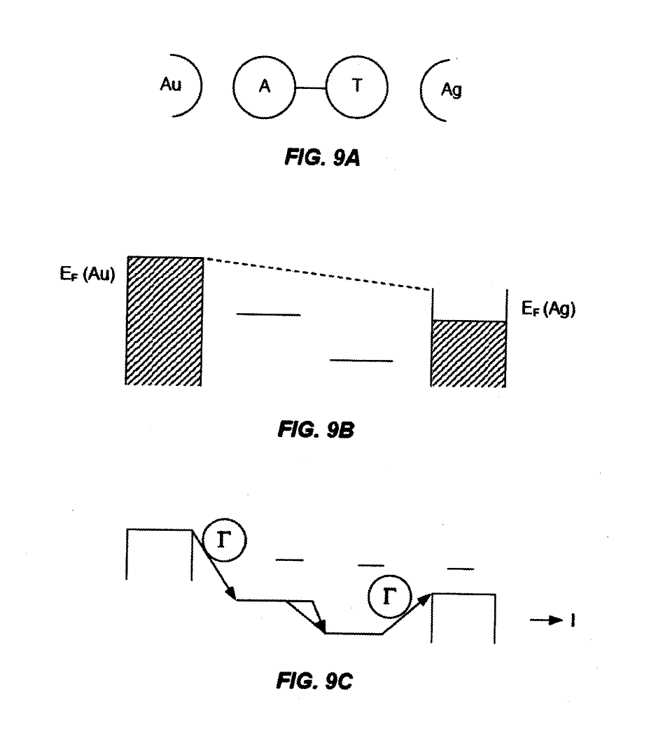

FIG. 9A depicts a nanoelectrode pair, wherein one nanoelectrode of the nanoelectrode pair is gold, and the other nanoelectrode pair is silver, and a nucleobase pair configured in a nanogap between the nanoelectrodes. FIG. 9B illustrates the Fermi level associated with removing an electron from the gold nanoelectrode E_{F}(Au) and the Fermi level associated with adding an electron to a silver nanoelectrode E_{F}(Ag) and the difference in the potentials of the Fermi levels E_{F}(Au) and E_{F}(Ag), as well as the HOMO level associated with the first (A) nucleobase and the HOMO-1 level associated with the second (T) nucleobase. FIG. 9C illustrates the changes in the energy of an electron as it is removed from the gold nanoelectrode with a Fermi level E_{F}(Au) to the A nucleobase utilizing a molecule-electrode coupling Γ₁, wherein the energy level of the electron (now associated with the A nucleobase) may be well aligned with the Fermi level E_{F}(Au) associated with the gold electrode. The electron is then transferred to the T nucleobase HOMO-1 level from the A nucleobase HOMO level, dropping to a lower energy state. This new energy state (now associated with the T nucleobase) may be well aligned with the Fermi level E_{F}(Ag) shift needed to transfer the electron to the silver nanoelectrode as a result of a molecule-electrode coupling Γ₂.

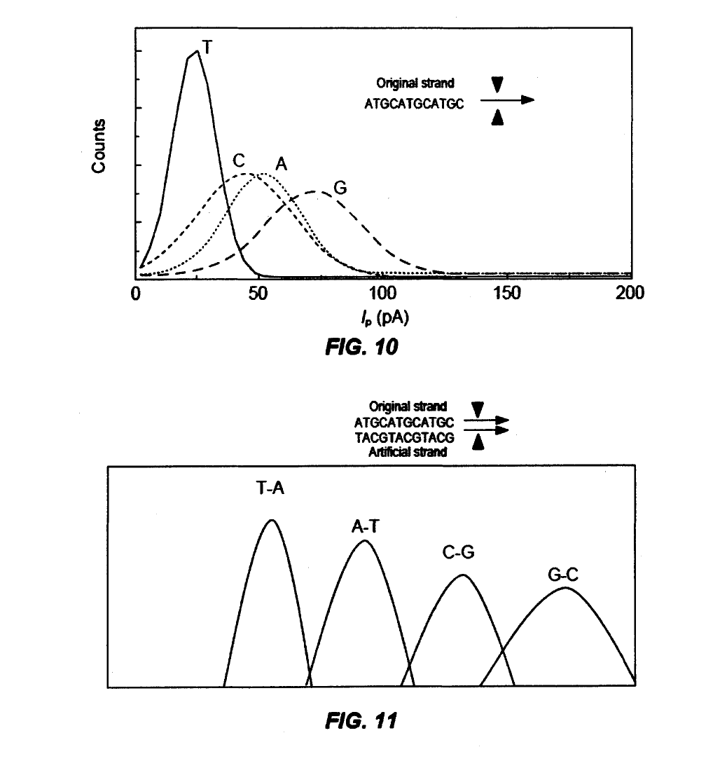

FIG. 10 depicts a histogram associated with the tunneling current distribution of four different natural DNA nucleobases of a single stranded DNA; it can be seen that the C and A nucleobases significantly overlap, such that many readings may be necessary in order to provide sequence information with high confidence levels.

FIG. 11 depicts a tunneling current histogram associated with a double stranded nucleic acid, wherein one strand utilizes modified nucleobases so as to provide better differentiation between the nucleobases, and the orientation of the nucleobases within the detecting nanoelectrode pair gap. The use of such base modifications may allow for improved confidence in providing nucleobase sequence or polymer structure information.

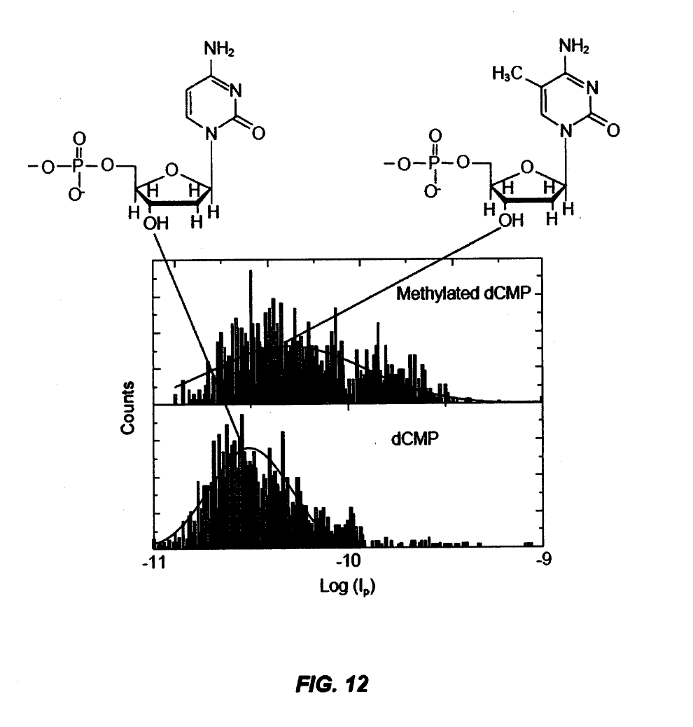

FIG. 12 depicts two histograms and molecular structures for different variants of dCMP, one is for natural dCMP, and the other is for methylated dCMP. Although there is overlap in the measured data, the peak associated with the different molecular structures is decidedly shifted. This difference provides at least two opportunities; one is to provide an approach for measuring naturally occurring methylated dCMP, while another is to permit the use of methylated bases in constructing a complementary strand to a single stranded nucleic acid, such that only bases of one strand may have methylated bases, so that the orientation of the strand as it translocates through the nanoelectrode pair gap may be determined.

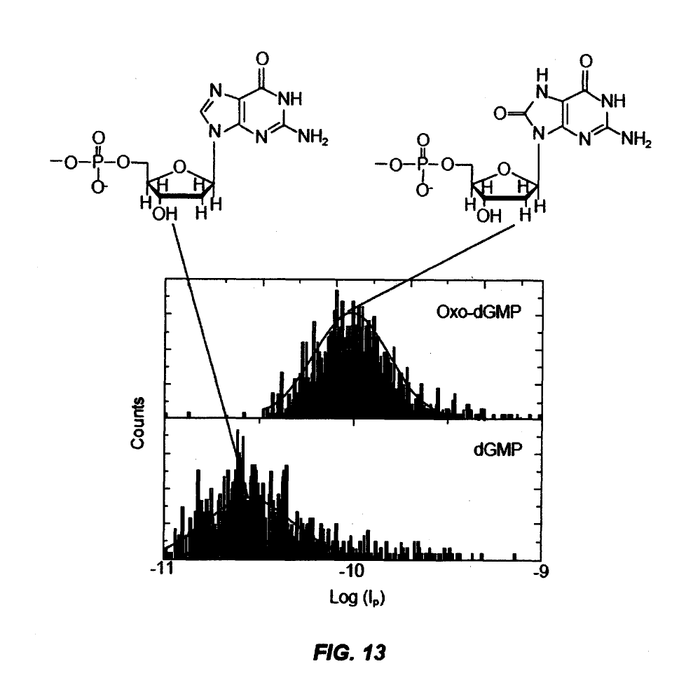

FIG. 13 depicts two histograms and molecular structures for different variants of dGMP, one is for natural dGMP, and the other is for 8-oxo-dGMP. Although there is overlap in the measured data, the peak associated with the different molecular structures is decidedly shifted. This difference provides an opportunity for utilizing 8-oxo-dGtp in constructing a complementary strand to a single stranded nucleic acid, such that only bases of one strand may have oxidized bases, so that the orientation of the strand as it translocates through the nanoelectrode pair gap may be determined.

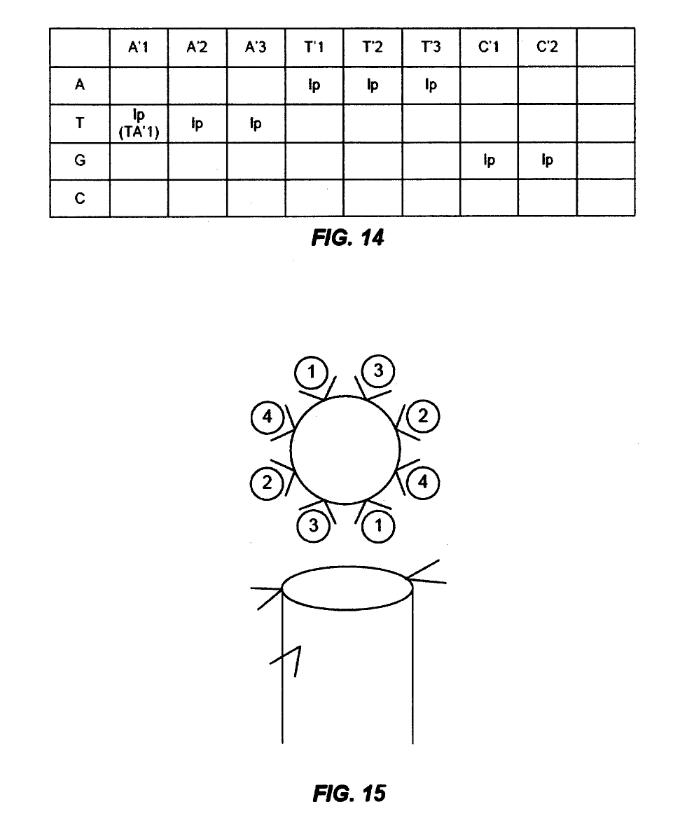

FIG. 14 depicts a table which depicts some of the options of combinations of base modifications wherein multiple different types of modified bases may be utilized, either singly or in combination such that there may be different types of A nucleobase modifications utilized at once be different types of G nucleobase modifications utilized at once, different types of C nucleobase modifications utilized at once, different types of T nucleobase modifications utilized at once, different types of uracil (U) nucleobase modifications utilized at once, or combinations thereof as may best provide sequence or polymer structure information with a desired confidence level.

FIG. 15 depicts a multi-electrode structure, wherein multiple different electrodes may be utilized, and the different electrodes may be of different metals or other materials so as to allow for different Fermi levels, so that during one or more translocations through the nanoelectrode structure, different materials with different Fermi levels may be utilized to make different determinations about the sequence or other aspects of the structure of a biopolymer.

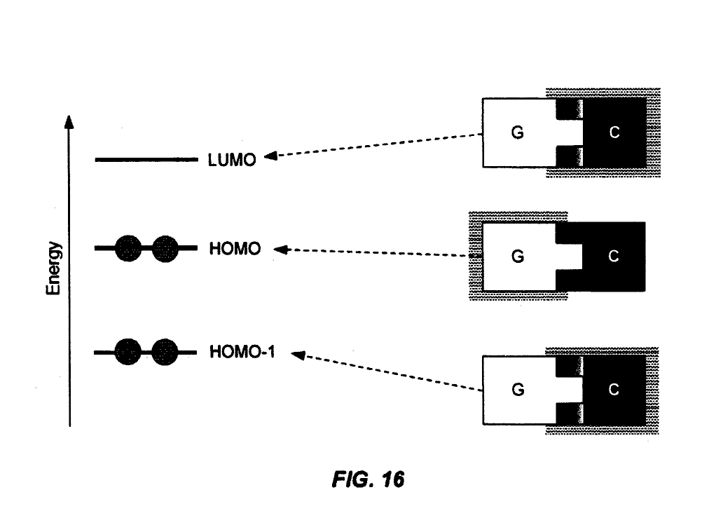

FIG. 16 depicts the energy levels in an energy diagram of the occupied and unoccupied orbitals of a GC nucleobase pair. The LUMO or lowest unoccupied molecular orbital is shown without any dots which may represent electrons which have occupied the orbital as a result of a tunneling current. The LUMO is shown in the uppermost depiction of the GC nucleobase as being associated with the C nucleobase, which may be a cytosine nucleobase as a cloud around the C nucleobase. The HOMO is shown with dots representing electrons in the middle depiction of the GC nucleobase as being associated with the G nucleobase, which may be a guanine nucleobase as a cloud around the G nucleobase. The HOMO-1 is shown with dots representing electrons in the lower depiction of the GC nucleobase as being associated with the C nucleobase, which may be a cytosine nucleobase as a cloud around the C nucleobase.

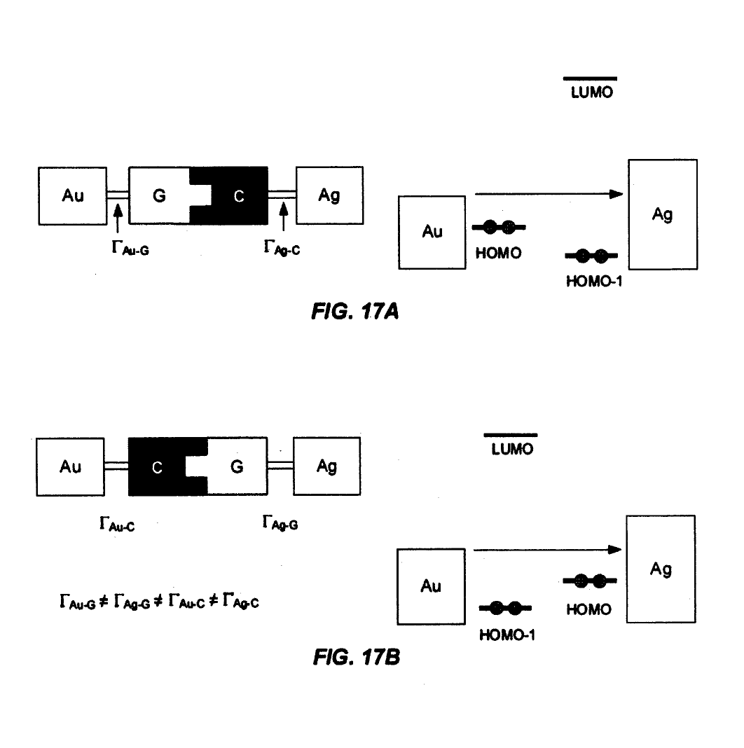

FIG. 17A depicts tunneling current passing from a gold nanoelectrode to a G nucleobase to a C nucleobase to a silver nanoelectrode. It further depicts two different molecule-electrode couplings, a first molecule-electrode coupling Γ_{Au-G} from the gold electrode to the G nucleobase, and a second molecule-electrode coupling Γ_{Ag-C} from the silver electrode to the C nucleobase. The electron is depicted as passing from the gold nanoelectrode to the HOMO energy level of the G nucleobase associated with a molecule-electrode coupling Γ_{Au-G}, then passing to a HOMO-1 energy level of the C nucleobase, and then passing to the silver nanoelectrode nucleobase associated with a molecule-electrode coupling Γ_{Ag-C}.

FIG. 17B depicts tunneling current passing from a gold nanoelectrode to a C nucleobase to a G nucleobase to a silver nanoelectrode. It further depicts two different molecule-electrode couplings, a first molecule-electrode coupling Γ_{Au-C} from the gold electrode to the C nucleobase, and a second molecule-electrode coupling Γ_{Ag-G} from the silver electrode to the G nucleobase. The electron is depicted as passing from the gold nanoelectrode to the HOMO-1 energy level of the C nucleobase associated with a molecule-electrode coupling Γ_{Au-C}, then passing to a HOMO energy level of the G nucleobase, and then passing to the silver nanoelectrode nucleobase associated with a molecule-electrode coupling Γ_{Ag-G}. It may be noted that Γ_{Ag-G} may not equal any of Γ_{Ag-C}, Γ_{Au-G}, or Γ_{Au-C}, and Γ_{Ag-C} may not equal either of Γ_{Au-G} or Γ_{Au-C}, and Γ_{Au-G} may not equal Γ_{Au-C}.

In some embodiments the single stranded DNA (ssDNA) template may be converted to double stranded DNA (dsDNA) by adding modified nucleotides for one or more of the base types using a polymerase. The dsDNA may then be denatured and in some embodiments the original template may be removed. The ssDNA may be sequenced using a tunneling current system. Similarly, an RNA template may be utilized with a reverse transcriptase to generate a ssDNA corresponding to the RNA template, and the ssDNA may be sequenced using a tunneling current system.

In other embodiments, the ssDNA may be converted to dsDNA by adding modified nucleotides for one or more of the base types using a polymerase, which may then be denatured and both stands may be sequenced utilizing a tunneling current system.

In further embodiments, the ssDNA may be converted to dsDNA by adding modified nucleotides for one or more of the base types using a polymerase, which may be sequenced directly as dsDNA utilizing a tunneling current system. The tunneling current system may utilize nanoelectrodes comprising different metals, particularly comprising different tip metals, so as to better differentiate base types and base orientations within the nanoelectrode pair.

In some embodiments, a single stranded Nucleic acid may be converted into a double stranded nucleic acid utilizing a reverse transcriptase (for converting a RNA molecule into a RNA paired with DNA double stranded nucleic acid) or a RNA polymerase may be utilized to convert a single stranded or double stranded DNA into a double stranded nucleic acid (for converting a DNA molecule into a DNA paired with RNA double stranded nucleic acid). The double stranded nucleic acid, which may comprise a whole natural nucleic acid, or a partly synthetic nucleic acid wherein some or all bases utilized in constructing the second strand of the double stranded nucleic acid may be modified bases, which may be natural bases (such as methylated guanosine), or synthetic bases such as bases with labels or tags.

In some embodiments dsDNA may be sequenced using nanoelectrode pairs wherein one tip of the nanoelectrode structure comprises one metal and a second tip of the nanoelectrode structure comprises a different metal. The metals may be selected such that the work functions cause a different signal for current flowing from the first nanoelectrode to the second nanoelectrode across a nucleobase pair (e.g., TA) to the second electrode than for current flowing from the first nanoelectrode to the second nanoelectrode across a reversed nucleobase pair (e.g., AT).

In some embodiments, multiple different metals may be utilized in several nanoelectrode pairs in a single nanochannel, such that a first one or more nanoelectrode pairs may be better suited to differentiating between a first set of nucleobases than a second set of nucleobases, while a second one or more nanoelectrode pairs may be better at determining a third set of nucleobases than a fourth set of nucleobases. The different sets of data from the different nanoelectrode pairs may be utilized to create a consensus determination of sequence with a higher confidence level than possible by utilizing the same number of nanoelectrode pairs wherein the metals comprising the tips may be the same.

In some embodiments a software algorithm may assume that all nucleobase measurements from a nanoelectrode pair result from a single orientation of the nucleic acid contained therebetween. In other embodiments a software algorithm may identify occasional orientation switches of the DNA relative to the nanoelectrodes, in which the nucleic acid strand which may be closest to a first nanoelectrode may switch such that it is closest to a second nanoelectrode of the nanoelectrode pair; the software algorithm may do such determination utilizing consensus either of data from the same strand obtained from different passes through the same nanoelectrode pair, or from other copies of the same sequence which may have been measured using other nanoelectrodes, or the same nanoelectrode pair. In other embodiments, a software algorithm may utilize several sets of electrode pairs in combination to make determinations as to the orientation of a strand relative to a particular pair of nanoelectrodes for a single strand without consensus. In still further embodiments, combinations of data from a single electrode may be used in combination with data from other data from the same electrode for the same nucleic acid strand, or may be combined with data from other nanoelectrode pairs in the same nanochannel, and or may be combined with data from other nanoelectrode pairs measuring the same DNA sequence in other nanochannels.

In some embodiments ssDNA may be converted to dsDNA by incorporating modified nucleotides for one or more of the base types using a polymerase, which may be a RNA or DNA polymerase. The dsDNA may then be sequenced using a tunneling current system. In some embodiments the tunneling system may have different metals on each nanoelectrode of the nanoelectrode pair. In further embodiments, different nanoelectrode pairs in a nanochannel may be configured to utilize several different metals, which may be utilized in different combinations for different nanoelectrode pairs.

In some embodiments a nanoelectrode pair may be fabricated utilizing a single surface metal and then a second metal may be added (for example by being electroplated onto one of the nanoelectrodes) in order to modify the work function of the tunneling measurement. In some embodiments each nanoelectrode of the nanoelectrode pair may have a different metal coated on each of the nanoelectrodes. In some cases, the second metal is different than the single surface metal. Alternative, the second metal is the same as the single surface metal.

In some embodiments the coating of one or more nanoelectrodes may be performed while monitoring of the electrode gap so as to determine whether the coating is of a desired thickness and or whether the nanogap is of a desired spacing, wherein the monitoring may be performed while plating a metal onto the nanoelectrode, or some material may be plated onto the nanoelectrode, and then a measurement may be made, at which time a determination as to whether the coating/plating process is complete, or whether an additional fixed period of coating/plating is needed, or a determination as to the duration for a coating/plating period may be determined.

The spacing of an electrode gap may be such that it is appropriate for detection using tunneling current for detection of single stranded DNA, or the spacing may be made larger such that the spacing is appropriate for detection of double stranded DNA, or the spacing may be made to be appropriate for any other desired biopolymer or other moiety.

In some embodiments the nanoelectrode pair that will be coated or plated may be fabricated at least in part by fabricating a trace, then breaking said trace, and then coating or plating one or more of the resulting electrode pairs.

In some embodiments, multiple nanoelectrode pairs in a single nanochannel may be used, with the spacing between the sets of nanoelectrode pairs such that, because of the dsDNA twist, the different nanoelectrode pairs measure known orientations of the dsDNA with each nanoelectrode pair. Natural B form dsDNA has a helix with a period of about 3.4 nm. If a pair of nanoelectrode pairs is spaced, for example, about 34 nm apart, the nanoelectrodes may nominally measure a dsDNA wherein the same orientation of the dsDNA is maintained with respect to nanoelectrodes. In other embodiments, for example, wherein a pair of nanoelectrode pairs is spaced about 99.5 nm apart, the nanoelectrodes may nominally measure the opposite strands of the dsDNA, allowing simultaneous monitoring of both orientations of the dsDNA within the nanochannel.

In some embodiments an adhesion layer may be utilized between the base electrode material, which may be silicon, silicon oxide, silicon nitrite, or other materials commonly utilized in semiconductor manufacturing, and the surface electrode material, which may be a metal or other conductor. In some embodiments the adhesion layer may be chromium, nickel-chromium, titanium, molybdenum and tungsten or other metals or oxides of metals commonly used as adhesion layers.

In some embodiments, a material which may comprise the nanoelectrode tip may be one or more of platinum, copper, silver, gold, Other metals, which may be noble metals or may be other types of metals and may be an allow of multiple metals, or may be a semiconductor, or may be another conductor such as a carbon nanotube, carbon buckyball or other nonmetallic, nonsemiconducting material.

In some embodiments a modified nucleobase which may be utilized may include nucleobases wherein the using Inocene, methyl modifications, thiol modifications to the nucleobases or other modifications. In some embodiments the modified nucleobases may be tunneling labeled nucleotides where the tunneling label is chosen to generate more unique tunneling current histograms than native nucleobases. In some embodiments the tunneling label may be covalently bound to the nucleobase itself. In other embodiments, the tunneling labels may be covalently bound or attached to the ribose, particularly to the 2′ position of the ribose such as 2′ methoxy, 2′ methoxyethoxy, 2′ aminoethoxy, or other similar modifications. In further embodiments, the ribose of a nucleobase may be modified such as in a LNA, BNA bicyclo-DNA, tricyclo-DNA, homo-DNA, or in other modifications of the ribose.

In some embodiments a system which may measure double stranded nucleobase sequences utilizing modified nucleobases and or nanoelectrode pairs with at least two different metals may be utilized to permit a simpler system, wherein a need for reduction of secondary structure may be reduced in comparison with a system which measures single stranded nucleic acids as a result of reduced secondary structure. In other embodiments, longer read lengths may result as a result of minimizing secondary structure, and resultant clogging of nanopores or nanochannels. In still other embodiments, the translocation speed of the nanopore or nanochannel system which measures double stranded nucleobases may be improved relative to a system which measures single stranded nucleobases, as a result of reduction of secondary structure, as a result of increased stiffness and or reduced interaction of different bases with different moieties associated with a surface which may otherwise interact with the nucleobases.

In some embodiments, the data resultant from measurement of tunneling current as measured by one or more pairs of nanoelectrode pairs may be improved as a result of utilizing modified nucleobases and or different metals with different Fermi levels for different nanoelectrodes, wherein the improvement may be an improved signal to noise of the measurements, or may be a larger separation in the centers of the peaks associated with the average or median tunneling current associated with measurement of the nucleobases or nucleobase pairs, or may be an improvement in the peak widths and or overlap of peaks of measured tunneling currents associated with different nucleobases or different nucleobase pairs.

In some embodiments, wherein one or more nanopores or nanochannels system may be clogged as a result of secondary structure and or interaction between two or more different strands of single or single stranded nucleic acid strands, one or more nucleases, which may be exonucleases or endonucleases may be utilized either singly or in combination with one or more restriction enzymes or other moieties which may result in the degradation of the clogging nucleic acid, thereby permitting further use of the one or more clogged nanopores and or nanochannels.

In some embodiments, the nanoelectrodes may be in part fabricated using electroplating or electrodeposition. A solution which comprises one or more metal salts may be provided such that fluidic contact may be made with all or a subset of the nanoelectrodes and an anode electrode, which may be a part of the fluidic system and or substrate structure, or may be part of an external device.

Individual nanoelectrodes or sets of nanoelectrodes may be electrically activated so as to serve as cathodes, wherein the metal salts may be reduced and thereby plated onto the electrically activated nanoelectrode or set of nanoelectrodes. The electrical activation may comprise applying a DC field between the anode and cathode electrodes wherein the DC field may be of a known voltage, and may be a fixed voltage, or may be a variable voltage.

The electrical activation may be for a fixed predetermined period of time, or the time may be determined by testing of, for example, the tunneling current generated utilizing the metal salts and the nanoelectrode pair wherein the anode may be electrically disabled while the tunneling current is being generated and measured.

The metal salt solution may be replaced with a different metal salt solution which comprises a different one or more metal salts. A different nanoelectrode or set of nanoelectrodes may be electrically activated so as to reduce the different one or metal salts, thereby electroplating a different set of one or more nanoelectrodes with a different one or more salts. By this process, any number of different nanoelectrodes may be plated with the desired different metals or different combinations of metals.

In some embodiments, the electroplating may be controlled so as to control the plating thickness, or to control the gap spacing of the different nanoelectrode pairs, wherein the different nanoelectrode pairs may have different gap spacings.

### Computer Systems

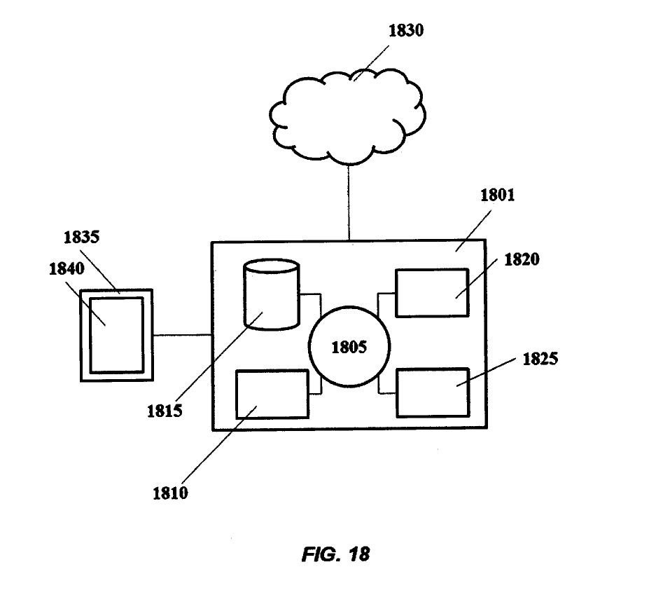

The present disclosure provides computer control systems that are programmed or otherwise configured to implement methods provided herein, such as calibrating sensors of the present disclosure. FIG. 18 shows a computer system 1801 that includes a central processing unit (CPU, also “processor” and “computer processor” herein) 1805, which can be a single core or multi core processor, or a plurality of processors for parallel processing. The computer system 1801 also includes memory or memory location 1810 (e.g., random-access memory, read-only memory, flash memory), electronic storage unit 1815 (e.g., hard disk), communication interface 1820 (e.g., network adapter) for communicating with one or more other systems, and peripheral devices 1825, such as cache, other memory, data storage and/or electronic display adapters. The memory 1810, storage unit 1815, interface 1820 and peripheral devices 1825 are in communication with the CPU 1805 through a communication bus (solid lines), such as a motherboard. The storage unit 1815 can be a data storage unit (or data repository) for storing data. The computer system 1801 can be operatively coupled to a computer network (“network”) 1830 with the aid of the communication interface 1820. The network 1830 can be the Internet, an internet and/or extranet, or an intranet and/or extranet that is in communication with the Internet. The network 1830 in some cases is a telecommunication and/or data network. The network 1830 can include one or more computer servers, which can enable distributed computing, such as cloud computing. The network 1830, in some cases with the aid of the computer system 1801, can implement a peer-to-peer network, which may enable devices coupled to the computer system 1801 to behave as a client or a server.

The CPU 1805 can execute a sequence of machine-readable instructions, which can be embodied in a program or software. The instructions may be stored in a memory location, such as the memory 1810. The instructions can be directed to the CPU 1805, which can subsequently program or otherwise configure the CPU 1805 to implement methods of the present disclosure. Examples of operations performed by the CPU 1805 can include fetch, decode, execute, and writeback.

The CPU 1805 can be part of a circuit, such as an integrated circuit. One or more other components of the system 1801 can be included in the circuit. In some cases, the circuit is an application specific integrated circuit (ASIC).

The storage unit 1815 can store files, such as drivers, libraries and saved programs. The storage unit 1815 can store user data, e.g., user preferences and user programs. The computer system 1801 in some cases can include one or more additional data storage units that are external to the computer system 1801, such as located on a remote server that is in communication with the computer system 1801 through an intranet or the Internet. The computer system 1801 can communicate with one or more remote computer systems through the network 1830.

Methods as described herein can be implemented by way of machine (e.g., computer processor) executable code stored on an electronic storage location of the computer system 1801, such as, for example, on the memory 1810 or electronic storage unit 1815. The machine executable or machine readable code can be provided in the form of software. During use, the code can be executed by the processor 1805. In some cases, the code can be retrieved from the storage unit 1815 and stored on the memory 1810 for ready access by the processor 1805. In some situations, the electronic storage unit 1815 can be precluded, and machine-executable instructions are stored on memory 1810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

The computer system 1801 can be programmed or otherwise configured to regulate one or more processing parameters, such as the substrate temperature, precursor flow rates, growth rate, carrier gas flow rate and reaction chamber pressure. The computer system 1801 can be in communication with valves between the storage vessels and a reaction chamber, which can aid in terminating (or regulating) the flow of a precursor to the reaction chamber.

Aspects of the systems and methods provided herein, such as the computer system 1801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Methods and systems of the present disclosure can be implemented by way of one or more algorithms. An algorithm can be implemented by way of software upon execution by the central processing unit 1805.

Devices, systems and methods of the present disclosure may be combined with and/or modified by other devices, systems, or methods, such as those described in, for example, JP 2013-36865A, US 2010/0025249, US 2012/0193237, US 2012/0322055, US 2013/0001082, US 2014/0300339, JP 2011-163934A, JP 2005-257687A, JP 2011-163934A and JP 2008-32529A, each of which is entirely incorporated herein by reference.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby. All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

1. A system for detecting a sample polymer, comprising:
an electrode structure, wherein the electrode structure includes at least one pair of nanoelectrodes and a nanogap between the nanoelectrodes, wherein the at least one pair of nanoelectrodes comprises a first electrode and a second electrode, the first electrode comprising a first conductive material and the second electrode comprising a second conductive material different from the first conductive material;
a voltage source that applies a voltage to the nanogap between the at least one pair of nanoelectrodes;
a translocation unit that moves the sample polymer through the nanogap between the pair of nanoelectrodes;
a measurement unit coupled to the at least one pair of nanoelectrodes, wherein the measurement unit measures electrical current passing through the sample polymer between the at least one pair of nanoelectrodes; and
a computer processor coupled to the measurement unit and programmed to determine an orientation and identity of monomers of the sample polymer relative to the nanoelectrodes in accordance with the electrical current measured with the measurement unit.

2. The system of claim 1, wherein the first conductive material has a Fermi level different from a Fermi level of the second conductive material.

3. The system of claim 1, wherein the first conductive material comprises gold and the second conductive material comprises silver.

4. The system of claim 1, wherein the first conductive material comprises platinum and the second conductive material comprises silver.

5. The system of claim 1, wherein the sample polymer is a biopolymer.

6. The system of claim 5, wherein the sample polymer comprises a double-stranded nucleic acid.

7. (canceled)

8. The system of claim 5, wherein the sample polymer has one or more modified base types incorporated in one of the strands of the sample polymer.

9. The system of claim 8, wherein the sample polymer includes one or more modified base types incorporated in one of the strands, wherein a molecule-electrode coupling for a modified base is different than for an unmodified base.

10. The system of claim 1, wherein a width of the nanogap between the pair of nanoelectrodes is less than a diameter of a sample polymer.

11. The system of claim 1, wherein the translocation unit is a pressure or electrokinetic source.

12. The system of claim 11, wherein the pressure source is a positive pressure source.

13. The system of claim 11, wherein the pressure source is a negative pressure source.

14. The system of claim 1, wherein the electrical current comprises tunneling current.

15. A method for detecting a sample polymer, comprising:
(a) subjecting the sample polymer to flow through a channel having an electrode structure, wherein the electrode structure includes at least one pair of nanoelectrodes and a nanogap between the nanoelectrodes, wherein the at least one pair of nanoelectrodes comprises a first electrode and a second electrode, the first electrode comprising a first conductive material and the second electrode comprising a second conductive material different from the first conductive material;
(b) applying a voltage to the nanogap between the at least one pair of nanoelectrodes;
(c) using a measurement unit coupled to the at least one pair of nanoelectrodes to measure electrical current passing through the sample polymer upon flow of the sample polymer through the channel and the nanogap; and
(d) using a computer processor to determine an orientation and identity of monomers of the sample polymer relative to the nanoelectrodes in accordance with the electrical current measured with the measurement unit.

16. The method of claim 15, wherein the first conductive material has a Fermi level different from a Fermi level of the second conductive material.

17. The method of claim 15, wherein the first conductive material comprises gold and the second conductive material comprises silver.

18. The method of claim 15, wherein the first conductive material comprises platinum and the second conductive material comprises silver.

19. (canceled)

20. (canceled)

21. (canceled)

22. (canceled)

23. (canceled)

24. The method of claim 15, wherein a width of the nanogap between the pair of nanoelectrodes is less than a diameter of a sample polymer.

25. The method of claim 15, wherein the translocation unit is a pressure or electrokinetic source.

26. (canceled)

27. (canceled)

28. The method of claim 15, wherein the electrical current comprises tunneling current.
